(19) United States
(12) Patent Application Publication
(10) Pub. No.: US 2007/0243245 A1
(43) Pub. Date: Oct. 18, 2007

(54) Oral Dosage Formulations, Methods of Preparing the Same, and Methods of Reducing Food Effects on Drug Release

(75) Inventor: Grant Wayne Heinicke, Rockaway, NJ (US)

Correspondence Address: CANTOR COLBURN, LLP, 55 GRIFFIN ROAD SOUTH, BLOOMFIELD, CT, 06002, US

(73) Assignee: ACTAVIS GROUP PTC HF, Hafnarfjordur (IS)

(21) Appl. No.: 11/736,081

(22) Filed: Apr. 17, 2007

Related U.S. Application Data
(60) Provisional application No. 60/792,443, filed on Apr. 17, 2006.

(51) Int. Cl. A61K 9/62

(52) U.S. Cl. 424/461

(57) ABSTRACT

A multi-particulate oral dosage form, comprising a plurality of pellets, the pellets comprising a core having disposed thereon a core composition layer, the core composition layer comprising an active agent, and a sustained-release coating disposed on the core composition layer, wherein the sustained-release coating comprises a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and about 10 wt % to about 50 wt % of colloidal silicon dioxide, based on the total weight of the alcohol-soluble material in the sustained-release coating. Methods of making the dosage form and methods of reducing food effects by administering the dosage form to a human subject are also disclosed.

### CROSS-REFERENCE TO RELATED APPLICATIONS

This application claims the benefits of U.S. Provisional Patent Application Ser. No. 60/792,443 filed Apr. 17, 2006, which is fully incorporated herein by reference.

### BACKGROUND

The food effect is a well-known phenomenon that can alter the pharmacokinetics of active agent distribution in the body. The food effect is defined as a differential absorption when an active agent is administered under fasted or non-fasted conditions. As a result of the food effect, many active agents should be taken either in fasted or non-fasted conditions to achieve the optimum effect. Examples of active agent dosage forms that have a food effect include carbamazepine tablets (to be taken with meals), captopril tablets (to be taken one hour before meals), or azithromycin tablets (to be taken 2 hours after a meal). Other drugs remain unaffected by food, for example, amoxicillin. Because the food effect can have a significant effect on pharmacokinetic parameters, the FDA recommends testing the bioequivalency of drug products either under fasted or non-fasted conditions, depending on the drug. Moreover, in testing under non-fasted conditions, the meal itself is standardized.

Diltiazem hydrochloride, used principally for its calcium channel blocking properties, finds application in the treatment of angina pectoris and hypertension. Because diltiazem has a short half-life in the bloodstream, frequent dosing or sustained-release dosage forms are preferred. It has been reported, however, that at least some diltiazem dosage forms exhibit a food effect.

Methylphenidate, [dl-threo-methyl-2-phenyl-2-(2-piperidyl)acetate], is the psychostimulant used most frequently in the treatment of hyperactivity and attention deficit disorder in children. Due to its short half-life, methylphenidate is usually given twice per day, for example, once after breakfast and once during the school day. Sustained-release forms of methylphenidate are thus desirable. A food effect has been reported for some methylphenidate formulations.

In view of the foregoing problems, there remains a need for improved dosage forms having reduced food effects.

### SUMMARY

A multi-particulate oral dosage form comprises a plurality of pellets, the pellets comprising a core having disposed thereon a core composition layer, the core composition layer comprising an active agent, and a sustained-release coating disposed on the core composition layer, wherein the sustained-release coating comprises a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and about 10 wt % to about 50 wt % of colloidal silicon dioxide, based on the total weight of the alcohol-soluble material in the sustained-release coating.

A method of making an oral dosage form comprises disposing a core composition layer on an inert core, the core composition layer comprising an active agent, and disposing a sustained-release coating on the core composition layer, wherein the sustained-release coating comprises a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and about 10 wt % to about 50 wt % of colloidal silicon dioxide, based on the total weight of the alcohol-soluble material in the sustained-release coating.

A method of reducing a food effect upon administration of a dosage form to a human subject comprising administering the disclosed multi-particulate dosage form to the human subject.

### BRIEF DESCRIPTION OF THE DRAWINGS

FIG. 1 is a dissolution profile in pH 6.8 phosphate buffer of one embodiment of methylphenidate pellets.

FIG. 2 is a dissolution profile in pH 6.8 phosphate buffer of one embodiment of tablets comprising methylphenidate pellets.

FIG. 3 is a dissolution profile in pH 6.8 phosphate buffer of one embodiment of diltiazem pellets and tablets comprising the pellets.

### DETAILED DESCRIPTION

An active agent is a species that, when administered to a patient, confers, directly or indirectly, a physiological effect on the patient. An indirect physiological effect, for example, includes the effect of a metabolite of the active agent. Active agent includes solvates (including hydrates) of the free compound or salt, crystalline and non-crystalline forms, as well as various polymorphs. For example, an active agent can include all optical isomers and all pharmaceutically acceptable salts thereof either alone or in combination. “Pharmaceutically acceptable salts” includes derivatives of the active agent, wherein the active agent is modified by making non-toxic acid or base addition salts thereof, and further refers to pharmaceutically acceptable solvates, including hydrates, of such compounds and such salts.

By “oral dosage form” is meant to include a unit dosage form prescribed or intended for oral administration. An oral dosage form comprises a plurality of subunits such as, for example, pellets, packaged for administration in a single dose such as a tablet, capsule, or sachet, for example. The oral dosage form optionally comprises a loading dose of the active agent in the form of, for example, a coating. A loading dose is an immediate release portion of the dosage form.

Dissolution profile as used herein, means a plot of the cumulative amount of active agent released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH” is meant a pH of about 1 to about 4. By the term “less acidic to basic pH” is meant a pH of greater than about 4 to about 7.5, preferably about 6 to about 7.5. A pH of about 1.2 can be used to simulate the pH of the stomach. A pH of about 6 to about 7.5, specifically about 6.8, can be used to simulate the pH of the intestine.

Release forms may also be characterized by their pharmacokinetic parameters. “Pharmacokinetic parameters” are parameters, which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

By “releasable form” is meant to include immediate-release, controlled-release, and sustained-release forms. Certain release forms can be characterized by their dissolution profile. By “immediate-release”, it is meant a conventional or non-modified release in which greater than or equal to about 70% of the active agent is released within 1 hour, specifically within 30 minutes of the initiation of dissolution. By “controlled-release” it is meant a dosage form in which the release of the active agent is controlled or modified over a period of time. “Sustained-release” or “extended-release” include the release of the active agent for an extended period of time so that the dosage frequency can be reduced.

Food is typically a solid food with sufficient bulk and fat content that it is not rapidly dissolved and absorbed in the stomach. In one embodiment, food is a meal, such as breakfast, lunch or dinner. In one embodiment, “with food”, “non-fasted” and “fed” are equivalent and mean that the dosage form is administered to a patient between about 30 minutes prior to about 2 hours after eating a meal.

The terms “without food” and “fasted” are equivalent. In one embodiment, fasted is defined as the condition of not having consumed solid food within about 1 hour prior or at least about 2 hours after such consumption. In another embodiment, fasted is defined as the condition of not having consumed solid food within about 1 hour prior to at least about 2 hours after such consumption.

For the purposes of biostudy, a fasted patient is defined as a patient who does not eat any food, i.e., fasts for at least 10 hours before the administration of a dosage form and who does not eat any food and continues to fast for at least 4 hours after the administration of the dosage form. The dosage form is administered with 180 ml of water during the fasting period, and water can be allowed ad libitum after 2 hours.

Also for the purposes of biostudy, a non-fasted patient is defined as a patient who fasts for at least 10 hours overnight and then consumes an entire test meal within 30 minutes of first ingestion. The dosage form is administered with 180 ml of water within 5 minutes after completion of the meal. No food is then allowed for at least 2 or 4 hours post-dose. Water can be allowed ad libitum after 2 hours. A high fat test meal provides approximately 800 to 1000 calories to the patient of which approximately 50% of the caloric content is derived from the fat content of the meal. A representative high fat high calorie test meal comprises 2 eggs fried in butter, 2 strips of bacon, 2 slices of toast with butter, 4 ounces of hash brown potatoes, and 8 ounces of whole milk to provide 150 protein calories, 250 carbohydrate calories, and 500 to 600 fat calories. High fat meals can be used in clinical bioequivalence and bioavailability studies of the active agent. High fat meals have been shown to alter absorption and uptake of some active agents.

The disclosed dosage form comprises a plurality of pellets, the pellets comprising a core comprising an active agent having disposed thereon a sustained-release coating composition that reduces the food effect of a dosage form comprising the pellets. Suitable pellets have a size of about 400 μm to about 2000 μm.

In one embodiment, the pellets comprise a core having disposed thereon a core composition layer comprising an active agent and optionally a binder. In one embodiment, the core composition layer is disposed directly on the surface of the core. Exemplary cores include inert spheroids, nonpareils, sugar spheroids, CELLETS®, CELPHERE®, microcrystalline cellulose spheres, spheres made of microcrystalline cellulose and one or more sugars, (e.g., lactose), and combinations comprising one or more of the foregoing cores. In one embodiment, the core is a sugar sphere. The size of cores may be, for example, about 250 μm to about 1500 μm. Commercially available sugar spheres are in US standard sieve size ranges of 14-16, 16-18, 18-20, 20-25, 25-30, 30-35, 40-60, for example. The cores comprise about 10 wt % to about 98 wt %, specifically about 20 wt % to about 90 wt %, and more specifically about 30 wt % to about 60 wt %, of the total weight of the pellets

The active agent-coated core may be formed by coating (e.g., spraying) the inert cores with an aqueous or non-aqueous suspension, which comprises the active agent. The active agent may be coated onto the inert cores in the presence of, for example, a binder, a filler, a solubilizer, and other additives, and combinations comprising one or more of the foregoing additives. The binder may be, for example, polyethylene oxide, hydroxypropylcellulose, hydroxypropylmethylcellulose, ethylcellulose, polyvinylpyrrolidone, cellulose acetate butyrate, hydroxypropylmethylcellulose phthalate, polyvinyl acetate phthalate, and the like, and combinations comprising one or more of the foregoing binders. The binder may comprise, for example, hydroxypropylcellulose, such as hydroxypropylcellulose NF 75-150 cps. The optional binder, when present, comprises about 0.1 wt % to about 20 wt %, specifically about 0.2 wt % to about 10 wt %, and more specifically about 3 wt % to about 8 wt %, of the total weight of the pellets. The suspension medium may comprise, for example, a solvent such as isopropyl alcohol, ethanol, methanol, water, and the like, and combinations comprising one or more of the foregoing solvents.

The core composition layer comprises an active agent such as diltiazem or methylphenidate, suitably in the form of a pharmaceutically acceptable salt. In one embodiment, the active agent is a water-soluble active agent. In some embodiments, the active agent is soluble in the dispersing agent employed in a rotary granulation process. Other suitable active agents include antacids, anti-inflammatory substances, coronary vasodilators, cerebral vasodilators, peripheral vasodilators, anti-infectives, psychotropics, antimanics, stimulants, anti-histamines, laxatives, decongestants, vitamins, gastro-intestinal sedatives, anti-diarrheal preparations, anti-anginal drugs, vasodilators, antiarrythmics, anti-hypertensive drugs, vasoconstrictors drugs useful to treat migraines, anticoagulants and antithrombotic drugs, analgesics, anti-pyretics, hypnotics, sedatives, anti-emetics, anti-nauseants, anticonvulsants, neuromuscular drugs, hyper- and hypoglycaemic agents, thyroid and antithyroid preparations, diuretics, antispasmodics, uterine relaxants, mineral and nutritional additives, antiobesity drugs, anabolic drugs, erythropoietic drugs, antiasthmatics, expectorants, cough suppressants, mucolytics, etc.

Additional suitable active agents include gastrointestinal sedatives such as metoclopramide and propantheline bromide; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotilline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; antispasmodic drugs such as dicyclomine and diphenoxylate; drugs affecting the rhythm of the heart, such as verapamil, nifedipine, diltiazem hydrochloride, procainamide, disopyramide, bretylium toxylate, quinidine sulfate and quinidine gluconate; drugs used in the treatment of hypertension such as propranolol hydrochloride, guanethidine mono-sulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicyclic acid, acetaminophen, codeine phosphate, codeine sulfate, oxydodone, dihydrocodeine tartrate, oxycodeinone, morphine, heroin, nalbuphine, butorphanol tartrate, pentazocine hydrochloride, cyclazacine, pethidine, buprenorphine, scopolamine and mefanamic acid; anti-epileptic drugs such as phenyloin sodium and sodium valproate; neuromuscular drugs such as dantrolene sodium; substances used in the treatment of diabetes such as tolbutamide, disbenase glucagon and insulin; drugs used in the treatment of thyroid gland disfunction such as triodothyronine, thyroxine and propylthiouracil; diuretic drugs such as furosemide, chlorthalidone, hydrochlorthiazide, spironolactone and trimterene; the uterine relaxant drug ritodrine; appetite suppressants such as fenfluramine hydrochloride, phentermine and diethylproprion hydrochloride; antiasthmatic and bronchodilator drugs such as aminophyline, theophyline, salbutamol, orciprenaline sulphate and terbutaline sulphate; expectorant drugs such as fuaiphenesin; cough suppressants such as dextromethorphan and noscapine; mucolytic drugs such as carbocisteine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; drug useful for treating Crohn's disease, e.g., 5-amino salicyclic acid, and the like.

In one embodiment, the active agent is present in the dosage form as a pharmaceutical salt. “Pharmaceutically acceptable salts” includes derivatives of the active agent, wherein the active agent is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

The active agent comprises about 2 wt % to about 85 wt %, more specifically about 10 wt % to about 70 wt %, of the total weight of the coated core, i.e., the core plus the core composition layer.

An active agent-binder coating mixture may be deposited on the core using a rotary granulation process. The active agent-binder coating mixture is atomized onto a fluidized bed of cores located in the rotor granulator. Because of the difference in size between the cores and the atomized active agent-binder mixture, the active agent sticks to the cores and the binder retains the active agent on the cores. In the fluidized bed, a rotor-disk granulator makes the cores move with fluid-like motion. As the cores move within the fluidized bed, they are sprayed with the active agent-binder mixture until the desired quantity of active agent is deposited upon the cores. Coating of the active agent and binder may optionally be followed by a drying step.

The core comprising the active agent is then coated with a sustained-release coating composition. The sustained-release coating composition comprises EUDRAGIT® RS and optionally EUDRAGIT® RL in a ratio of about 50:50 to about 100:0, specifically about 50:50 to 99:1, and more specifically about 75:25 to about 98:2. EUDRAGIT® RS is a copolymer of acrylic and methacrylic esters comprising quaternary ammonium groups and having a ratio of quaternary ammonium groups to neutral meth(acrylic) esters of 1:40. EUDRAGIT® RL is a copolymer of acrylic and methacrylic esters comprising quaternary ammonium groups and having a ratio of quaternary ammonium groups to neutral meth(acrylic) esters of 1:20. The EUDRAGIT® RS and EUDRAGIT® RL together comprise about 20 wt % to about 90 wt % of the total weight of the sustained-release coating. The sustained-release coating composition also comprises about 10 wt % to about 50 wt % of colloidal silicon dioxide, specifically about 20 wt % to about 40 wt % of colloidal silicon dioxide, based on the total weight of alcohol-soluble material in the sustained-release coating composition. The alcohol-soluble material in the coating composition includes the EUDRAGIT® RS and EUDRAGIT® RL.

The sustained-release coating composition optionally comprises a filler such as, for example, talc, kaolin, calcium sulfate, and combinations comprising one or more of the foregoing fillers. In one embodiment, the optional filler comprises talc. When present, the amount of optional filler is about 10 wt % to about 200 wt %, specifically about 20 wt % to about 100 wt % of the weight of the alcohol-soluble material in the sustained-release coating composition.

The sustained-release coating optionally comprises a plasticizer. The plasticizer can be water-soluble or water-insoluble. Exemplary water-soluble plasticizers include triethylcitrate, triacetin, polyethylene glycol, propylene glycol, sorbitol, glycerin, and combinations comprising one or more of the foregoing plasticizers. Exemplary water-insoluble plasticizers include dibutyl sebacate, diethyl phthalate, dibutyl phthalate, tributyl citrate, acetyl tributyl citrate, castor oil, mineral oil, glyceryl monostearate, and combinations comprising one or more of the foregoing plasticizers. The plasticizer comprises about 0 wt % to about 30 wt %, specifically about 5 wt % to about 15 wt %, of the total weight of the copolymers of acrylic and methacrylic esters in the coating composition.

The sustained-release coating composition can be applied to the core using a coating technique used in the pharmaceutical industry, such as fluid bed coating. Once applied and dried, the alcohol-soluble fraction of the sustained-release coating comprises about 5 wt % to about 35 wt % of the total weight of the pellets, or about 7 wt % to about 25 wt % of the total weight of the pellets.

The sustained-release coating may be dried before applying an optional second coating. A color imparting agent may be added to the sustained-release coating composition, or a rapidly dissolving seal coat containing color may be coated over the sustained-release coating layer provided that the seal coat is compatible with and does not affect the dissolution of the sustained-release coating layer. Exemplary film-forming agents include hydroxypropyl methylcellulose, hydroxypropylcellulose, polyethylene oxide, polyvinylpyrrolidone, polyethylene glycol, and combinations comprising one or more of the foregoing film-forming agents.

The multi-particulate dosage form of the active agent is optionally encapsulated in hard gelatin to provide a desired quantity of active agent in an oral dosage form. Alternatively, the multi-particulate dosage form may be made into tablets, for example, by first adding about 35 wt % to about 80 wt %, specifically about 50 wt % to about 70 wt %, of a solid pharmaceutically acceptable tablet excipient which will form a compressible mixture with the pellets and which may be formed into a tablet without crushing the coated cores. The tablets optionally comprise an effective amount of a tablet disintegrating agent and a lubricant. The solid pharmaceutically acceptable tablet excipient may comprise, for example, carnuba wax, lactose, dextrose, mannitol, microcrystalline cellulose, kaolin, powdered sucrose, and combinations comprising one or more of the foregoing excipients. The tablet disintegrant may comprise crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, hydrogenated vegetable oil (e.g., Lubritab®), zinc stearate, and combinations comprising one or more of the foregoing lubricants.

In some embodiments, the pharmacokinetic profile of the active agent in the disclosed dosage form is not substantially affected by the non-fasted or fasted state of a subject (e.g., a mammal such as a human) ingesting the compositions. This means that there is no substantial difference in the quantity of drug absorbed or the rate of drug absorption when the dosage forms are administered in the non-fasted versus the fasted state, that is, administration of the composition to a mammalian subject. The drug absorption may be measured as Cₘₐₓ, AUC₀₋ₜ, AUC_{0-∞}, or a combination comprising one or more of the foregoing pharmacokinetic parameters. In one embodiment, the drug absorption is measured as Cₘₐₓ, AUC₀₋ₜ, and AUC_{0-∞}.

The invention thus encompasses an active agent dosage form in which administration of the composition to a subject in a fasted state is bioequivalent to administration of the composition to a subject in a non-fasted state. The difference in Cₘₐₓ, AUC₀₋ₜ, AUC_{0-∞}, or a combination comprising one or more of the foregoing pharmacokinetic parameters for the active agent dosage form, when administered in the non-fasted versus the fasted state, is less than about 20%, less than about 10%, or less than about 5%. In one embodiment, the difference in Cₘₐₓ, AUC₀₋ₜ, and AUC_{0-∞}for the active agent dosage form, when administered in the non-fasted versus the fasted state, is less than about 20%, less than about 10%, or less than about 5%.

In one embodiment, administration of the active agent dosage form to a subject in a fasted state is bioequivalent to administration of the composition to a subject in a non-fasted state, wherein bioequivalency is established by a 90% Confidence Interval of 0.80 to 1.25 for AUC and Cₘₐₓ when administered to a human subject. In another embodiment, administration of the active agent dosage form to a subject in a fasted state is bioequivalent to administration of the composition to a subject in a non-fasted state, wherein bioequivalency is established by a 90% Confidence Interval of 0.80 to 1.25 for AUC and Cₘₐₓ when administered to a human subject.

### EXAMPLES

The following examples further illustrate the invention but, of course, should not be construed as in any way limiting its scope. In particular, the processing conditions are merely exemplary and can be readily varied by one of ordinary skill in the art.

### Example 1

This example illustrates the production of an exemplary active agent oral dosage formulation.

| | |
|---|---|
| Sugar spheres: | 1000 g |
| Methylphenidate Hydrochloride: | 440 g |
| Polyethylene oxide (PEO): | 22 g |
| Alcohol: | 1230 g |
| Water: | 370 g |

PEO was dissolved in the alcohol water mixture. After dissolution of PEO the methylphenidate was added. Sugar spheres were charged into a rotor granulator and the active agent solution was sprayed onto the sugar spheres to produce API-containing cores.

A coating solution was made as follows.

| | |
|---|---|
| EUDRAGIT ® RS: | 85.2 g |
| EUDRAGIT ® RL: | 16.1 g |
| Triethyl citrate: | 11.5 g |
| Sodium lauryl sulfate: | 1.1 g |

All coating components were dissolved in ethanol (749 g). To the ethanolic solution were added:

| | |
|---|---|
| Talc: | 41 g |
| Colloidal SiO₂: | 27.3 g |

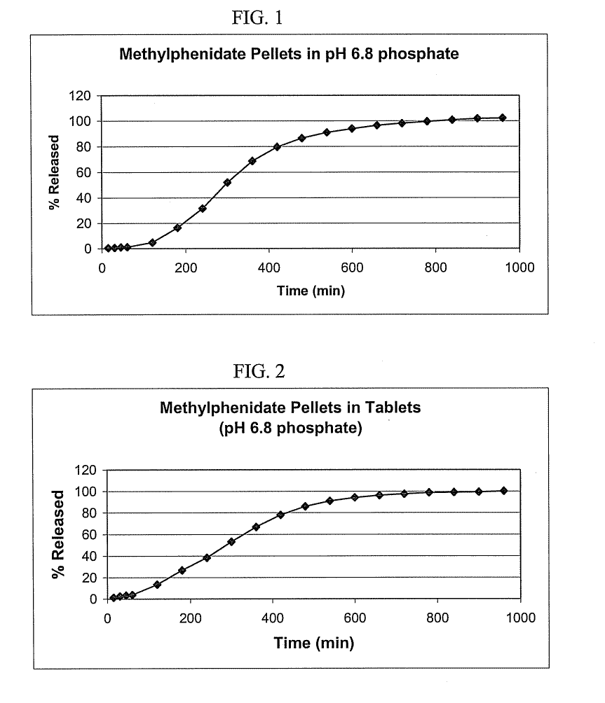

The resulting suspension was sprayed onto 700 grams of the methylphenydate-containing cores to produce pellets with the release profile shown in FIG. 1 (Drug release measured in USP Apparatus II, pH 6.8 phosphate buffer, 100 rpm).

The pellets can be placed into hard gelatin capsules each containing a nominal quantity of methylphenidate. Alternatively, 1000 g of these pellets were blended with:

| | | |
|---|---|---|
| Carnuaba wax: | 726 | g |
| Lubritab: | 597 | g |
| Ac-Di-Sol ®: | 59.7 | g |
| Cab-O-Sil ®: | 3.8 | g |

and compressed into tablets of nominal weights to provide therapeutic amounts of methylphenidate. Drug release from representative compressed tablets are presented in FIG. 2.

### Example 2

A solution of hydroxypropyl cellulose (HPC) was prepared in ethanol. Diltiazem was suspended in the solution and applied to sugar spheres to a potency of 68%. A solution of PEO in ethanol/water was prepared. Diltiazem was suspended in the solution and applied to 5 kg of these cores until the final potency was 85%.

The overall composition of the cores is presented in the Table below.

Diltiazem Core Formulation

| Item # | Ingredient | Grams | Wt % |
|---|---|---|---|
| 1 | Sugar Sphere (30–35 mesh) | 1076.0 | 8.7 |
| 2 | Polyethylene Oxide (PEO) | 593.0 | 4.7 |
| 3 | Hydroxypropyl Cellulose | 204.0 | 1.6 |
| 4 | Diltiazem HCl | 10620.0 | 85.00 |
| | Total | 12493.0 | 100.00 |
| 1 | Ethanol | 20000.0 | |
| 2 | Water | 3500.0 | |

To two separate lots of 3 kg of these cores was sprayed different quantities of a polymer solution with suspended solids which was prepared as follows

Coating Composition for the Cores - 3 Kgs

| | | | Grams, | Grams, |
|---|---|---|---|---|
| ITEM # | INGREDIENTS | % | batch 1 | batch 2 |
| | Total Polymer Solids | | | |
| 1 | EUDRAGIT ® RS PO | 75.00 | 170 | 415 |
| 2 | EUDRAGIT ® RL PO | 15.00 | 34 | 83 |
| 3 | Triethyl Citrate | 9.00 | 20 | 50 |
| 4 | Sodium Lauryl Sulfate | 1.00 | 2 | 5 |
| | Total | 100.00 | | |
| | Solids added after dissolving | | | |
| | Polymer (Calculated as 50% | | | |
| | of Polymer Solids) | | | |
| 5 | Colloidal Silicon dioxide | 20.00 | 45 | 110 |
| 6 | Talc | 30.00 | 65 | 165 |
| | Total | 50.00 | | |

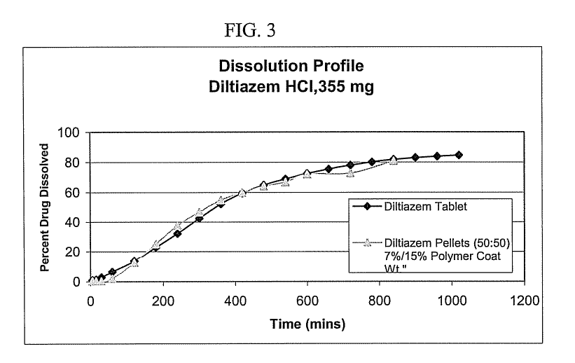

The batch 1 (about 7 wt % coating weight) and batch 2 (about 15 wt % coating weight) pellets were blended in a 1:1 ratio. The resulting blend had the dissolution profile shown in FIG. 3, measured in a USP Apparatus-2, pH 6.8 (phosphate buffer), 900 ml, 100 rpm, 37° C. The pellets were placed into hard gelatin capsules each containing 355 mg of diltiazem hydrochloride. Alternatively, the pellets were tableted as follows:

Tablet composition for 355 mg Diltiazem Sustained Release Tablets

| ITEM # | INGREDIENTS | %/Tablet | Grams |
|---|---|---|---|
| 1 | Polymer Coated Diltiazem HCl Pellets- | 46.8 | 513.7 |
| | Blended | | |
| 2. | Carnauba Wax-powder | 23.1 | 253.2 |
| 3 | Hydrogenated Vegetable Oil (Lubritab) | 9.6 | 105.5 |
| 4 | Microcrystalline Cellulose (Avicel 102) | 15.5 | 169.8 |
| 5 | Sodium Carboxymethylcellulose | 5 | 54.9 |
| | (AC-Di-Sol ®) | | |
| | Total | 100 | 1097 |

Disclosed herein are multi-particulate dosage forms suitable for sustained-release of an active agent. The particles can be advantageously employed in capsule dosage forms as well as tablets. The novel sustained-release coating provides dosage forms with substantially no food effect.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A multi-particulate oral dosage form, comprising a plurality of pellets, the pellets comprising a core having disposed thereon a core composition layer, the core composition layer comprising an active agent, and a sustained-release coating disposed on the core composition layer, wherein the sustained-release coating comprises
a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and
about 10 wt % to about 50 wt % of colloidal silicon dioxide, based on the total weight of the alcohol-soluble material in the sustained-release coating.

2. The dosage form of claim 1, wherein the first polymer and the second polymer are present in a ratio of about 75:25 to about 98:2.

3. The dosage form of claim 1, comprising about 20 wt % to about 40 wt % of the colloidal silicon dioxide based on the total weight of the alcohol-soluble material in the sustained-release coating.

4. The dosage form of claim 1, wherein the sustained-release coating further comprises about 1 wt % to about 30 wt % of a plasticizer, based on of the total weight of the copolymers of acrylic and methacrylic esters in the coating composition.

5. The dosage form of claim 1, wherein the core composition layer further comprises a binder.

6. The dosage form of claim 1, wherein the core comprises a sugar sphere.

7. The dosage form of claim 1, wherein the active agent comprises diltiazem hydrochloride or methylphenidate hydrochloride.

8. The dosage form of claim 1, wherein upon administration of the active agent dosage form to a human subject in the fasted state compared to the non-fasted state, there is less than about a 20% difference in the AUC, the Cₘₐₓ, or both.

9. The dosage form of claim 1, wherein administration of the active agent dosage form to a human subject in a fasted state is bioequivalent to administration of the composition to a human subject in a non-fasted state, wherein bioequivalency is established by a 90% Confidence Interval of 0.80 to 1.25 for AUC or Cₘₐₓ.

10. A method of making an oral dosage form, comprising
disposing a core composition layer on an inert core, the core composition layer comprising an active agent, and
disposing a sustained-release coating on the core composition layer, wherein the sustained-release coating comprises
a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and
about 10 wt % to about 50 wt % of colloidal silicon dioxide, based on the total weight of the alcohol-soluble material in the sustained-release coating.

11. The method of claim 10, wherein the first polymer and the second polymer are present in a ratio of about 75:25 to about 98:2.

12. The method of claim 10, comprising about 20 wt % to about 40 wt % of the colloidal silicon dioxide, based on the total weight of the alcohol-soluble material in the sustained-release coating.

13. The method of claim 10, wherein the sustained release coating further comprises about 1 wt % to about 30 wt % of a plasticizer, based on of the total weight of the copolymers of acrylic and methacrylic esters in the coating composition.

14. The method of claim 10, wherein the core composition layer further comprises a binder.

15. The method of claim 10, wherein the core comprises a sugar sphere.

16. The method of claim 10, wherein the active agent comprises diltiazem hydrochloride or methylphenidate hydrochloride.

17. A method of reducing a food effect upon administration of a dosage form to a human subject, comprising administering a multi-particulate dosage form comprising a plurality of pellets, the pellets comprising a core having disposed thereon a core composition layer, the core composition layer comprising an active pharmaceutical ingredient, and a sustained-release coating disposed on the core composition layer, wherein the sustained-release coating comprises
a first polymer comprising a copolymer of acrylic and methacrylic esters comprising quaternary ammonium groups and having a ratio of quaternary ammonium groups to neutral meth(acrylic) esters of 1:40 and optionally a second polymer comprising a copolymer of acrylic and methacrylic esters comprising quaternary ammonium groups and having a ratio of quaternary ammonium groups to neutral meth(acrylic) esters of 1:20, wherein the ratio of the first polymer to the second polymer is about 50:50 to about 100:0, and wherein the first and second polymer comprise about 20 wt % to about 90 wt % of the total weight of the sustained-release coating; and
about 10 wt % to about 50 wt % of colloidal silicon dioxide, based on the total weight of the alcohol-soluble material in the sustained-release coating.

18. The method of claim 17, wherein upon administration of the dosage form to a human subject in the fasted state compared to the non-fasted state, there is less than about a 20% difference in the AUC, the Cₘₐₓ, or both.

19. The method of claim 17, wherein upon administration of the dosage form to a human subject in the fasted state compared to the non-fasted state, there is less than about a 10% difference in the AUC, the Cₘₐₓ, or both.

20. The method of claim 17, wherein administration of the dosage form to a human subject in a fasted state is bioequivalent to administration of the composition to a human subject in a non-fasted state, wherein bioequivalency is established by a 90% Confidence Interval of 0.80 to 1.25 for AUC or Cₘₐₓ.
